(19) United States
(12) Patent Application Publication
(10) Pub. No.: US 2010/0054575 A1
(43) Pub. Date: Mar. 4, 2010

(54) Analysis method for 5-differential complete blood cell based on visual image

(76) Inventors: Honghua Zhou, Jiangxi (CN); Hong Dai, Jiangxi (CN); Xiao Yan, Jiangxi (CN); Jia Yang, Jiangxi (CN)

Correspondence Address: TRASKBRITT, P.C., P.O. BOX 2550, SALT LAKE CITY, UT, 84110, US

(21) Appl. No.: 12/311,751

(22) PCT Filed: Sep. 6, 2007
(86) PCT No.: PCT/CN2007/002665

§ 371 Date: Nov. 12, 2009

(30) Foreign Application Priority Data
Oct. 13, 2006 (CN) 200610124771.7

(51) Int. Cl. G01N 33/49

(52) U.S. Cl. 382/134

(57) ABSTRACT

An analysis method for five-differential complete blood cell based on visual image is provided. The analysis method for five-differential complete blood cell is carried out in two channels. The method includes: counting blood cells by typical cytometry to obtain the amount of blood cells; identifying and counting the blood cells by image recognition to obtain the percent of each kind of blood cells; then calculating the results from the two channels to obtain the number of each kind of blood cells.

### CROSS-REFERENCE TO RELATED APPLICATIONS

This is a national phase entry under 35 U.S.C. §371 of International Patent Application PCT/CN2007/002665, filed Sep. 6, 2007, published in Japanese as International Patent Publication WO 2008/046292 A1 on Apr. 24, 2008, which claims the benefit under 35 U.S.C. §119 of Chinese Patent Application CN 200610124771.7 filed Oct. 13, 2006.

### FIELD OF THE INVENTION

The present invention relates to an analysis method for blood cells, especially relating to an analysis method for five-differential complete blood cell.

### BACKGROUND OF THE INVENTION

A blood cell analyzer is medical testing equipment with integration of light, mechanics, electronics and software. It belongs to the medical testing equipment in the medical equipment industry. It is mainly used for analysis of a human's complete blood cells to replace the previous routine blood examination. It has the advantages of fast detection, simple operation and more detection parameters, and so on. According to the number of white blood cell differential, the blood cell analyzer is divided into a three-differential blood cell analyzer and a five-differential blood cell analyzer.

The blood cell differential analysis method is the basis of the design and manufacture of blood cell analyzers. Based on different blood cell differential analysis methods, both domestic and abroad manufacturers have designed and manufactured different types of blood cell differential instruments.

The five-differential blood cell analyzer was invented in the mid 1990s. In 1981, Technicon designed the first H 6000 five-differential blood cell analyzer based on flow cytometry, and in 1985, it presented the formed H 1 blood cell counting system.

In the 1990s, the companies of Coulter, Abbott, Sysmex, ABX and the like, on the basis of a three-differential blood cell counting instrument and combining the methods of histochemistry, immunohistochemistry, high-frequency transduction, laser light scattering and the like, presented a series of five-differential multi-parameter blood cell analyzer systems. For instance, STKS II, a five-differential blood cell analyzer of US Coulter Company, is based on the combination of an impedance method, a conductance method, and a light scattering method, which are pure physical methods; five-differential blood cell analyzer Advia 120 of US Abbott Company is based on the laser scattering and cytochemical staining method; five-differential blood cell analyzer SF-3000 of Japan Sysmex Company is based on the combination of laser flow cytometry detection and cell group chemistry staining method; its SE-9000 is based on the multi-channel impedance and radio frequency detection method; while the XE-2100 is based on the combination of the laser flow cytometry detection method and the nucleic acid fluorescence staining method. The five-differential blood cell analyzer Pentra60 of France ABX Company is based on the combination of DHSS, dual-sheath flow system, cytochemical staining method, and an optical analysis method, while its Pentra120retic is based on the combination of a laser flow cytometry method, a fluorescence staining method, a sheath flow impedance method and a light scattering method. The five-differential blood cell analyzer CD-3700R of US ABBOTT Company is based on the MAPSS method (multi-angle polarized scatter separation method) and has four-angle light-scattering.

Similar to the three-differential blood cell analyzer, the five-differential blood cell analyzer is designed and manufactured on the basis of the existing blood cell differential analysis methods. Their grouping (classification) methods both indirectly simulate a variety of electrical signals generated by the cells through a mathematical model and work out the result of cell classification. The results will also be affected by the accuracy of the collection of cell electrical signals, the rationality of the mathematical model and many other factors. The rate of accuracy is only 85%˜90%, so about 10% of the samples need to be smeared and stained for artificial microscopic reexamination.

For the above reasons, the artificial smear staining microscopic examination is proposed by the international committee for standardization in hematology (ICSH) as the reference method for the analysis of complete blood cells. Coulter, Sysmex and Roche companies have developed dyeing machines with an auto push function, as auto push and stain the samples that need reexaminations to reduce the workload of reexaminations. However, this does not change the complete white blood cell analyzer in methodology, it just improves the pre-process before artificial microscopic examination on original basis, and this does not completely solve the full automation problem of the five-differential complete blood cell analyzer, and it is still greatly affected by human factors.

As for the instruments for sorting simple cells through the image analysis method, the 8200, which was produced by Hitachi, Ltd. in Japan in the 1980s, is representative. Such instruments completely use the image analysis method, staining blood smear, scanning each field of vision with a microscope with the scanning lens, and then comparing the obtained cell image with standard image stored in the instrument to analyze and finally determine the cell type. Such a pure image analysis method is limited by the calculating speed of the computer, and cannot enter the field of practical application.

At present, in the classification of blood cell morphology, a five-differential or more-differential classification, or even the blood cell analyzer with the special naive cell analysis channels, cannot fundamentally solve the problem of blood cell morphology, and cannot entirely replace the artificial classification with the results of instrument classification.

### SUMMARY OF THE INVENTION

The purpose of the present invention is to provide a new analysis method for a five-differential complete blood cell based on visual image, so that the five-differential complete blood cell analysis can achieve the effect of artificial microscopic examination and classification.

The present invention can be achieved through the following ways: combining the image recognition technology with the conventional cell counting technology, and identifying and classifying the blood cells according to the standard reference method of the International Committee for Standardization in Hematology (ICSH).

To be more specific, a five-differential complete blood cell can be divided into two channels: one is a blood cell counting channel, which adopts the conventional blood cell counting method to count the blood cells in a blood sample to obtain the total amount of blood cells; another is an image identifying and counting channel of the blood cells, which adopts image recognition technology to identify and calculate the blood cells from the same blood sample to identify and calculate the percentage of various types of blood cells. Then, it calculates the counting result of the blood cell counting channel and image identifying and calculating result of the blood cells to obtain the number of the various types of blood cells in the blood samples.

The blood cell counting channel described in the present invention can set two branch channels, i.e., white blood cell counting channel and red blood cell/platelet counting channel. In general, if the white blood cells need to be counted, then set the white blood cell counting channel; if red blood cells/platelets need to be counted, then set the red blood cell/platelet counting channel; if the white blood cells and the red blood cells/platelets need to be counted at the same time, then set the white blood cell counting channel and the red blood cell/platelet counting channel at the same time.

In the present invention, it can also set hemoglobin measurement in the white blood cell counting channel so as to add hemoglobin measurement items.

The blood cell counting channel described in the present invention can use the conventional blood cell counting method, i.e., it can use the conventional impedance method, and can also adopt the conventional flow cytometry method. In other words, the blood cell counting channel described in the present invention can be achieved through the counting method of the existing typical three-differential blood cell analyzer.

The blood cell counting channel composed of the typical three-differential blood cell analyzer described in the present invention can be achieved through the following ways: first, the blood samples are diluted according to the proportion in conformity with the white blood cell count, and then divided into two branch channels; add hemolytic agents in one channel, and then perform count/color comparison to obtain the result of white blood cell counting and hemoglobin measurement; another channel is diluted for the second time, and the red blood cells and platelets are counted to obtain the counting result of red blood cells and platelets.

The blood cell image identifying and counting channel described in the present invention can also set two branch channels, i.e., a white blood cell image identifying and counting channel and a red blood cell/platelet image identifying and counting channel. In general, if white blood cells need to be image identified and calculated, then set the white blood cell image identifying and counting channel; if red blood cell/platelet needs to be image identified and calculated, then set red blood cell/platelet image identifying and counting channel; if the white blood cell, red blood cell/platelet need to be image identified and calculated at the same time, then set white blood cell image identifying and counting channel and red blood cell/platelet image identifying and counting channel at the same time.

For the white blood cell image recognition and calculation in the blood cell image identifying and counting channel in the present invention, identifying and counting 100 to 800 of the white blood cells in the blood sample is one image recognition and calculation unit of image recognition and calculation. However, this range is not to limit the present invention. While meeting the accuracy of statistical results and ensuring sufficient image recognition speed of the white blood cells or the requirement for image recognition speed of the white blood cells is not very high, the amount of image recognition and calculation unit of the white blood cells can be wider. In the present invention, 400 to 600 white blood cells are the best.

For the red blood cell image recognition and calculation of the blood cell image identifying and counting channel in the present invention, identifying and counting 1,000 to 20,000 of the red blood cells in the blood samples is one image recognition and calculation unit of image recognition and calculation. However, this range is not to limit present invention. While meeting the accuracy of statistical results and ensuring sufficient image recognition speed of the red blood cells or the requirement for image recognition speed of the red blood cells is not very high, the amount of image recognition and calculation unit of the red blood cells can be wider. In the present invention, 8,000 to 12,000 red blood cells are the best.

The white blood cell image identifying and counting channel described in the present invention can be achieved through the following ways: first, preparing the blood smear, staining, and then performing a micrograph, and finally performing white blood cell image recognition and calculation.

The white blood cell image recognition and calculation described in the present invention can be aimed at the following several types of white blood cells: neutrophil cells, acidophilic cells, basophilic cells, lymphocytes, monocytes, naive granular cells and other abnormal white blood cells not included in the above cells. However, the types of white blood cells described in the present invention are not to limit the method in the present invention, while, if possible, abnormal white blood cells can also be further classified.

Based on the white blood cell classification described in the present invention, the white blood cells can be divided into neutrophil cells, acidophilic cells, basophilic cells, lymphocytes, and monocytes according to the existing white blood cell five-differential classification standard. The five kinds of cells is image identified and calculated, while the identification and calculation of the naive granular cells and other abnormal white blood cells are included in other cells.

The red blood cell/platelet image identifying and counting channel described in the present invention can be achieved through the following ways: first, performing vital staining, preparing a blood smear, staining, and then performing micrography, and performing red blood cell/platelet image recognition and calculation.

For the red blood cell/platelet image identifying and counting channel described in the present invention, the micrography, image recognition and calculation of red blood cells/platelets can be aimed at the following types: red blood cells, platelets, reticulocytes, nucleated red blood cells, stem cell images, and abnormal red blood cells. However, the classification of blood cells described in the present invention is not to limit the method in the present invention; if possible, the classification of blood cells can also be further divided.

The red blood cell/platelet image recognition and calculation described in the present invention include platelet identifying and counting, red blood cell identifying and counting, reticulocyte image recognition, nucleated red blood cell image recognition, stem cell image recognition, abnormal red blood cell image recognition, and the percentage calculation of platelet and red blood cells.

According to the method described in the present invention, a new five-differential complete blood cell analysis equipment (or system) can be constructed based on visual image.

The five-differential complete blood cell analyzer according to the present invention includes the following units: blood cell counting unit, blood cell identifying and counting unit, and result output unit.

The units of the five-differential complete blood cell analyzer according to present invention can be further described as follows:

The blood cell counting unit of the five-differential complete blood cell analyzer according to the present invention includes white blood cell counting, hemoglobin measurement, red blood cell counting and platelet counting. The white blood cell counting includes the dilution of blood samples, adding hemolytic agents, blood cell counting; hemoglobin measurement includes the dilution of blood samples, adding hemolytic agents, hemoglobin index measurement; red blood cell counting and platelet counting also include the dilution of blood samples, respective red blood cell and platelet counting.

The blood cell image identifying and counting unit of the five-differential complete blood cell analyzer according to the present invention includes two channels. One channel is a white blood cell image identifying channel, including the preparation of the blood smear, staining, micrography, five-differential white blood cell image identifying and counting, naive granular cell image identifying and counting, abnormal white blood cell image identifying and counting, and the percentage calculation of various white blood cells. Another channel is a red blood cell/platelet image identifying channel, including the vital staining of blood samples, smearing, micrography, platelet and red blood cell identifying, counting and percentage calculation, reticulocyte image identifying, nucleated red blood cell image identifying, stem cell image identifying, and abnormal red blood cell image identifying.

The result output unit described in the present invention is to calculate the total amount of white blood cells output from the blood cell counting unit and the percentage of various white blood cells output from the blood cell image identifying unit of the same blood sample to obtain specific value of various white blood cells in testing blood samples and to calculate the total amount of red blood cells output from the blood cell counting unit and the percentage of platelets/red blood cells output from the blood cell image identifying unit of the same blood sample to obtain specific value of platelet in testing blood samples based on image. So the five-differential complete blood cell analysis can achieve the effect of artificial microscopic examination and classification.

The five-differential complete blood cell analyzer described in the present invention also includes the blood sample introducing unit. Blood sample introducing can adopt automatically introducing and can also adopt manual introducing. The automatic introducing unit can include automatic transmission of blood samples, barcode identification, mixing, puncture sampling, and separating blood through shear valve.

The present invention combines the image identification technology with conventional cell counting technology, and imitates the standard reference method of the International Committee for Standardization in Hematology (ICSH) to identify and classify white blood cells. It provides a new five-differential complete blood cell analysis system based on visual image. It enables the five-differential complete blood cell analysis to achieve the effect of artificial microscopic examination and classification, which greatly improves the accuracy of blood cell classification results and the automation level of complete blood cell analysis.

### DESCRIPTION OF DRAWINGS

FIG. 1 is a schematic block diagram of the present invention, including a sample introducing unit, a blood cell counting unit, a white blood cell identifying and counting unit, a red blood cell/platelet identifying and counting unit, and a result output unit.

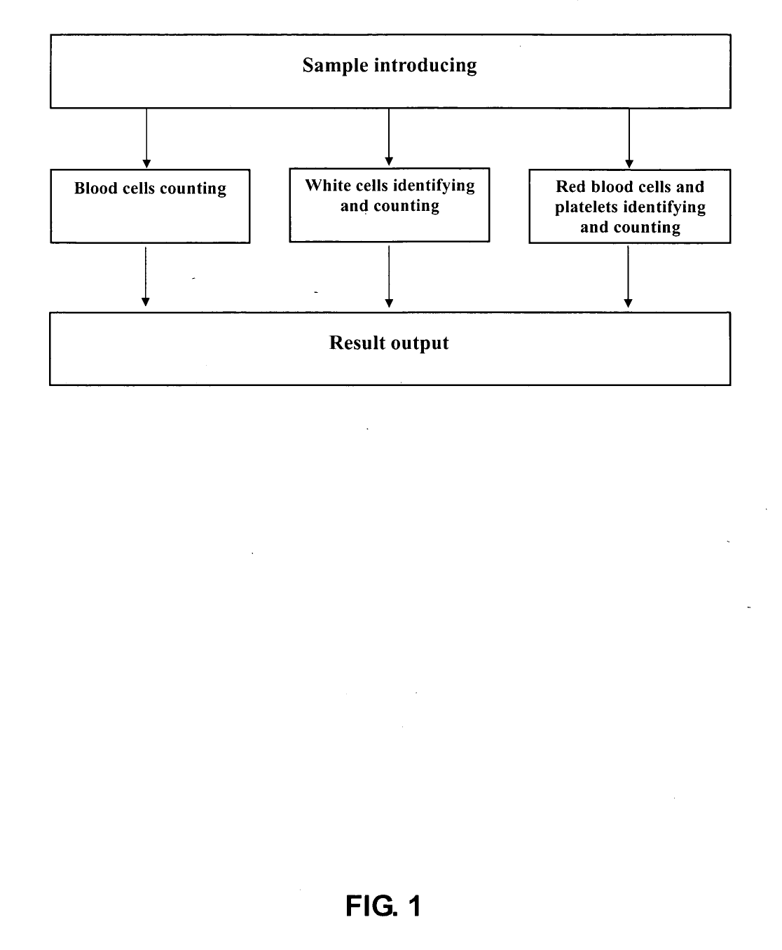

FIG. 2 is a schematic diagram of an embodiment of the present invention. (1) the sample introducing unit, including automatic introducing and manual introducing, (2) the blood cell counting and hemoglobin measuring unit, (3) the white blood cell identifying and counting unit, (4) the red blood cell, platelet identifying and counting unit, and (5) the result output unit.

### DETAILED DESCRIPTION OF THE INVENTION

The present invention will be further described through the following embodiments:

### Embodiment

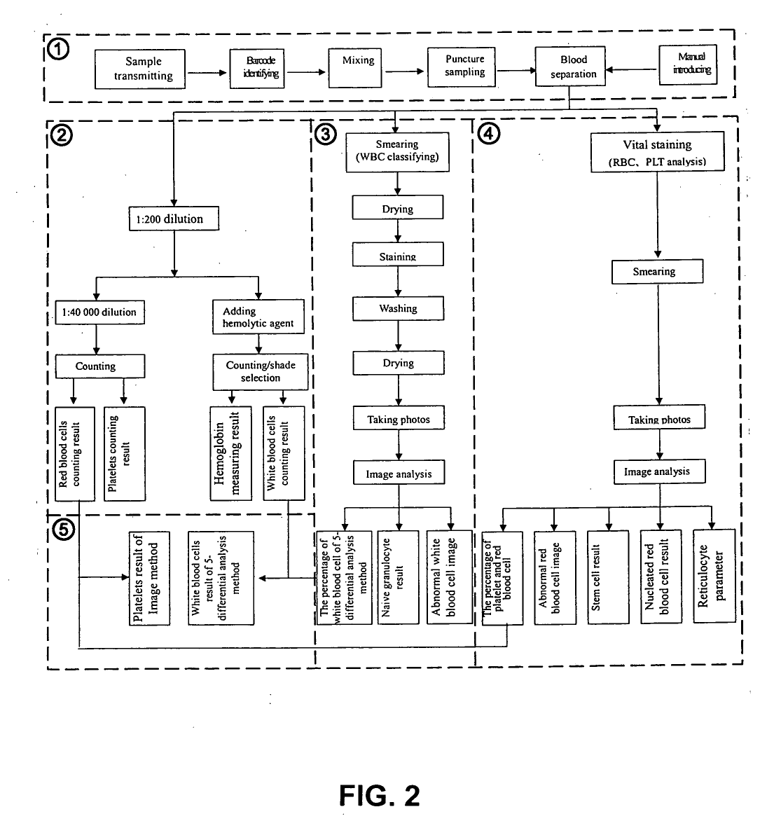

FIG. 2 is a schematic diagram of the specific embodiment.

This embodiment includes the sample introducing unit, the blood cell counting unit, the blood cell identifying and counting unit, and the result output unit. The blood cell counting unit includes hemoglobin measurement; the blood cell identifying and counting unit includes two branch channels: white blood cell identifying and counting, red blood cell and platelet identifying and counting, the two branch channels.

The sample introducing unit in the present embodiment adopts the combination of automatic sample introducing and manual sample introducing, wherein the automatic sample introducing includes: the automatic introduction of blood samples, barcode identification, mixing, puncture sampling, and blood separation valve. In manual sample introducing, the sample is manually introduced and, after the instrument sampling, the blood separation valve will automatically separate the blood.

The blood cell counting and hemoglobin measuring unit in the present embodiment is divided into two branch channels: one is the red blood cell and platelet counting channel and the other is the white blood cell counting and hemoglobin measurement channel. The process is: dilute the blood samples in the ratio of 1:200, and then a part of diluted blood enters the red blood cell and platelet counting channel, and then the blood sample entered this channel is diluted in the ratio of 1:40,000, perform red blood cell counting and platelet counting respectively, and respectively output the result of the red blood cell counting and platelet counting; add hemolytic agents to the blood sample entered the white blood cell counting and hemoglobin measurement channel firstly, and then perform the white blood cell counting and colorimetric measurement of hemoglobin, and respectively output the result of white blood cell counting and hemoglobin measurements.

The function of the blood cell counting and hemoglobin measuring unit in the present embodiment is actually a function implemented by a conventional three-differential blood cell analyzer. In other words, the blood cell counting and hemoglobin measuring unit in the present embodiment is implemented by adopting the method of the existing three-differential blood cell analyzer.

The white blood cell identifying and counting unit in the present embodiment includes: blood smear staining, micrography, image identifying and counting. After the blood smear is stained, the red blood cells are removed, then micrography is taken, and finally, image identifying and counting is performed. Image identifying and counting mentioned here includes: the division of white blood cells, feature extraction, feature classification and counting. Mainly through the identification of white blood cells, the white blood cells can be divided into neutrophil cells, acidophilic cells, basophilic cells, lymphocytes, monocytes, naive granular cells and abnormal white blood cells. Calculate the identified various cells separately and calculate total of them, and 400 cells are set as an identifying and counting unit to calculate the percentage of various types of cells.

The red blood cell/platelet identifying and counting branch unit in the present embodiment includes: vital stain of blood sample, preparation of smear, micrography, and image identifying and counting. After vital stain of the blood sample, prepare the blood smear, then take micrography, and finally perform image identifying and counting. Image identifying and counting mentioned here includes: the division of red blood cells, feature extraction, feature classification and counting. Mainly through the identification of red blood cells, the red blood cells can be divided into reticulocytes, nucleated red blood cells, stem cells, and abnormal red blood cells. In the meantime, calculate the percentage of platelets and red blood cells and set 10,000 red blood cells as an identifying and counting unit to calculate the percentage of platelets and red blood cells.

The result output unit in the present embodiment will gather partial results obtained from the blood cell counting and hemoglobin measuring unit, the white blood cell identifying and counting branch unit, and the red blood cell/platelet identifying and counting branch unit. In detail, white blood cell count results obtained from the blood cell counting and hemoglobin measuring unit multiplied by the percentage of various cells obtained from the white blood cell identifying and counting branch unit is the number of various white blood cells in the blood samples. The red blood cell/platelet count results obtained from the red blood cell/platelet counting unit multiplied by the percentage of platelets and the red blood cells obtained from the platelet identifying and counting unit is the number of platelets in the blood samples based on image identifying.

At the same time, the present embodiment can also obtain the measurement result of hemoglobin, the identifying result of naive granular cell images, the image identifying result of abnormal white blood cells, the image identifying result of reticulocytes, the image identifying result of nucleated red blood cells, the image identifying result of stem cells, and the image identifying result of abnormal red blood cells.

1. An analysis method for five-differential complete blood cell based on visual image includes blood cells counting, wherein, it sets two channels of blood cell counting and blood cell image identifying and counting, the percentage of various types of blood cells obtained by the blood cell image identifying and counting channel and the total amount of various types of blood cells obtained by the blood cell counting channel are calculated to obtain the value of various types of blood cells.

2. The method according to claim 1, wherein it respectively sets white blood cells counting and red blood cells/platelets counting channels in blood cells counting channels.

3. The method according to claim 2, wherein it adds a new measurement unit of hemoglobin in white blood cell counting channels.

4. The method according to claim 1, wherein it respectively sets white blood cells image identifying and counting channels and red blood cells/platelets image identifying and counting channels in blood cells image identifying and counting channels.

5. The method according to claim 1, wherein identifying and counting 100-800 of white blood cells in a blood sample is an image identifying and counting unit for white blood cells image identifying and counting channel.

6. The method according to claim 5, wherein identifying and counting 400-600 of white blood cells in a blood sample is an image identifying and counting unit for the white blood cells image identifying and counting channel.

7. The method according to claim 1 wherein identifying and counting 1,000-20,000 of red blood cells in a blood sample is an image identifying and counting unit for the red blood cells image identifying and counting channel.

8. The method according to claim 7, wherein identifying and counting 8,000-12,000 of red blood cells in a blood sample is an image identifying and counting unit for red blood cells image identifying and counting channel.

9. The method according to claim 1 wherein a white blood cells image identifying and counting channel includes preparation of blood smear, staining, micrography, image recognition and calculation; red blood cell image identifying and counting channel includes blood vital staining, preparation of blood smear, micrography, image recognition and calculation.

10. The method according to claim 9, wherein a white blood cells image identifying and counting channel includes neutrophil cells, acidophilic cells, basophilic cells, lymphocytes, monocytes, naive granulocyte, and abnormal white cells.

11. The method according to claim 9, wherein a red blood cells image identifying and counting channel includes platelets, reticulocytes, nucleated red blood cells, stem cells, and abnormal red blood cells.

12. A method for analyzing a complete blood cell using a five-part differential system, the method comprising:
counting blood cells in a sample to obtain a total amount of blood cells therein; and
identifying and counting the blood cells in the sample using image recognition and calculation to calculate a percentage of various types of blood cells.

13. The method of claim 12, further comprising calculating a number of the various types of blood cells using the total amount of blood cells and the percentage of various types of blood cells.

14. The method of claim 12, wherein counting blood cells in a sample to obtain a total amount of blood cells therein comprises utilizing a white blood cell counting channel and a red blood cell counting channel.

15. The method of claim 12, wherein identifying and counting the blood cells in the sample using image recognition and calculation comprises identifying and counting at least one of platelets, red blood cells, reticulocytes, nucleated red blood cells, stem cells, and abnormal red blood cells.

16. The method of claim 15, wherein identifying and counting the blood cells in the sample using image recognition and calculation to calculate a percentage of various types of blood cells comprises calculating a percentage of at least one of platelets and red blood cells in the blood sample.

17. The method of claim 12, wherein identifying and counting the blood cells in the sample using image recognition and calculation to calculate a percentage of various types of blood cells comprises calculating a percentage of at least one of neutrophil cells, acidophilic cells, basophilic cells, lymphocytes, monocytes, naive granulocyte, and abnormal white cells.

18. The method of claim 12, wherein identifying and counting the blood cells in the sample using image recognition and calculation to calculate a percentage of various types of blood cells comprises identifying and counting the blood cells in the sample using a white blood cell channel and a red blood cell channel.

19. The method of claim 12, wherein identifying and counting the blood cells in the sample using image recognition and calculation to calculate a percentage of various types of blood cells comprises:
preparing a blood smear;
staining the blood smear;
performing a micrograph; and
performing blood cell image recognition and calculation.

20. A method of analyzing a blood sample using a five-part differential, the method comprising:
determining a total number of blood cells in the blood sample;
determining a percentage of various types of blood cells in the blood sample using image recognition; and
calculating a number of each of the various types of blood cells in the blood sample from the total number of blood cells in the blood sample and the percentage of various types of blood cells in the blood sample.
